(19) United States
(12) Patent Application Publication
(10) Pub. No.: US 2024/0158811 A1
(43) Pub. Date: May 16, 2024

(54) Noninvasive AAV Vectors For Highly Efficient Gene Delivery To The Nervous System

(71) Applicant: Temple University-Of The Commonwealth System of Higher Education, Philadelphia, PA (US)

(72) Inventor: SHUXIN Li, Wynnewood, PA (US)

(21) Appl. No.: 18/549,948

(22) PCT Filed: Mar. 11, 2022
(86) PCT No.: PCT/US2022/020005

§ 371 Date: Sep. 11, 2023

Related U.S. Application Data
(60) Provisional application No. 63/159,502, filed on Mar. 11, 2021.

(51) Int. Cl. C12N 15/86; A61K 48/00; C07K 14/005

(52) U.S. Cl. CPC C12N 15/86; A61K 48/0058; C07K 14/005; C12N 2310/113; C12N 2310/13; C12N 2750/14143; C12N 2830/008

(57) ABSTRACT

The present invention generally relates to engineered adeno-associated viral (AAV) vectors capable of traversing the blood-brain barrier and transducing specific neural cells with high efficiency. The present invention also relates to methods of producing engineered AAV vectors and treating diseases and disorders associated with neural cell damage and degeneration.

### CROSS-REFERENCE TO RELATED APPLICATIONS

This application claims priority under 35 U.S.C. § 119(e) to U.S. Provisional Patent Application Ser. No. 63/159,502, filed Mar. 11, 2021, the disclosure of which is incorporated herein by reference in its entirety.

### BACKGROUND OF THE INVENTION

A major challenge in neuroscience research is the ability to deliver target genes to specific types of neural cells widely distributed in the central nervous system (CNS). CNS gene transfer is widely used for gene expression (upregulation, knockdown, and editing), neuronal circuit modulation, in vivo neural cell imaging, disease model development, and molecular therapies. Invasive injections of viral vectors are frequently employed for in vivo gene transduction that targets regional cells, but regional gene delivery is usually insufficient to target neural cells widely distributed in the entire CNS, such as damaged cells in neurotrauma, Alzheimer's, Parkinson's, Huntington's, and amyotrophic lateral sclerosis. Neurological diseases, including traumatic brain injury and stroke, may partially and temporally open BBB, but traversing BBB remains a major challenge for efficient gene delivery to the CNS and neurotherapeutics. Transgenic animals are commonly used to study CNS function, but they have numerous disadvantages, including that results may be confounded by compensatory mechanisms and cannot be translated directly to the clinic.

Most AAV serotypes fail to cross the blood-brain barrier (BBB) in adult mammals although several (e.g., AAV9 & AAVrh8) pass BBB with low efficiency. The low AAV transduction efficacy in adult CNS after systemic administration is the main limit on its wide application in neuroscience research and treatments for neurological diseases. Capsid proteins have a highly conserved eight stranded (β-barrel core and expansive loops with connected β-strands and unique surface features. Because the BBB hinders invasion of blood-borne viruses into CNS, increased BBB crossing and neural tropism require optimizing AAV capsid proteins. As the features of AAV capsids are essential for interacting with vasculature and for crossing BBB, engineered AAV9 capsids can enhance the ability to cross BBB and transduce CNS cells. Engineering AAV9 capsids by targeted mutagenesis and insertions of peptide residues has improved transduction in CNS, but they usually transduce neural cells only in some CNS regions and the overall transgene expression levels are low in most targeted cells.

Furthermore, axon disconnections in CNS usually cause a devastating functional loss, and people who suffer CNS axonal injury may lose the essential ability for daily life, such as paralysis, inability to use their hands, and blindness. Lesioned axons in adult mammalian CNS fail to regenerate and there are no effective regenerative strategies to treat patients with CNS axon injuries. It thus is very important to develop highly effective strategies to regenerate CNS axons that are damaged in traumatic injury and other neurological disorders. CNS neurons lose the ability to regenerate axons with age, and this limits functional recovery after CNS injury. Several cell-autonomous molecules, including PTEN, SOCS3, cAMP, and Kruppel-like factors, play significant roles in controlling the growth ability of mature neurons. However, none of these approaches were translated to clinics yet and there is a persistent need to identify better targets and improved delivery methods. The best targets are probably those with the potential to impact multiple genes simultaneously. Some miRNAs, including let-7, that target multiple genes are critical for controlling axon regeneration in CNS, which makes them attractive targets for CNS repair.

Let-7 miRNA is very important for regulating age-dependent decline in axon regeneration in worms. In C. elegans, let-7, a 22-nucleotide miRNA, is upregulated during development and is critical in controlling the age-dependent loss of axon growth capacity. Let-7 mutants exhibit enhanced axon regeneration in adult worms. As a highly conserved RNA-binding protein expressed during embryogenesis, lin28 plays crucial roles in regulating cell functions in development, pluripotency, and metabolism by inhibiting let-7 biogenesis and enhancing translation of mRNAs for several metabolic enzymes. In C. elegans, let-7 inhibits regeneration by downregulating lin41, a protein that controls cell growth by suppressing let-7 expression and miRNA-mediated gene silencing. Therefore, the lin28/let-7/lin41 signaling pathway is responsible for the age-related decline of axon regeneration in worms.

Finally, because multiple factors contribute to axon growth failure after CNS injury, successful regenerative strategies probably require combinatorial treatments that target different responsible mechanisms, including the non-permissive environment. The major in vivo approach to surmount strong inhibition by CSPGs is to digest them by local application of chondroitinase ABC (ChABC). However, ChABC has serious shortcomings that may preclude its use as a treatment in patients. Problems include incomplete removal of inhibitory components from CSPGs, the short life of enzymatic activity at 37° C., and the inability of ChABC to cross the blood-brain barrier.

Thus, there is a need in the art for improved compositions and methods for efficient and specific delivery of therapeutics to the CNS to treat axonal damage and degeneration. This invention satisfies this unmet need.

### SUMMARY OF THE INVENTION

In one aspect, the present invention relates, in part, to an engineered adeno-associated viral (AAV) vector comprising: a) an engineered AAV viral capsid; and b) a modified viral genome, comprising a nucleic acid sequence encoding one or more therapeutic molecule.

In some embodiments, the engineered AAV viral capsid comprises a phenylalanine at amino acid residue 446 of wild-type AAV9 capsid protein VP1, a phenylalanine at amino acid residue 731 of wild-type AAV9 capsid protein VP1, an aspartic acid at amino acid residue 587 of wild-type AAV9 capsid protein VP1, a glycine at amino acid residue 588 of wild-type AAV9 capsid protein VP1, a TLAVPFK (SEQ ID NO: 2) insertion after amino acid residue 588 of wild-type AAV9 capsid protein VP1, or any combination thereof. In one embodiment, the wild-type AAV9 capsid protein VP1 comprises an amino acid sequence of SEQ ID NO: 1. In one embodiment, the engineered AAV viral capsid comprises an amino acid sequence of SEQ ID NO: 3.

In one embodiment, the modified viral genome further comprises a neural cell-specific promoter. In one embodiment, the neural cell-specific promoter limits expression to one or more type of cell selected from the group consisting of: neurons, astrocytes, oligodendrocytes, and microglia. In one embodiment, the neural cell-specific promoter is one or more selected from the group consisting of: synapsin I (Syn), glial fibrillary acidic protein (GFAP), myelin basic protein (MBP), and CD68.

In one embodiment, the one or more therapeutic molecule of the modified viral genome comprises one or more inhibitor of one or more neural anti-regenerative pathway targets.

In one embodiment, the one or more neural anti-regenerative pathway targets is one or more selected from the group consisting of: let-7 miRNA, and one or more receptor of chondroitin sulfate proteoglycans (CSPGs).

In one embodiment, the one or more inhibitor of let-7 miRNA is one or more selected from the group consisting of: anti-let-7 tough decoy (TuD) RNA (anti-let-7 TuD), lin28, and lin41.

In one embodiment, the one or more inhibitor of one or more receptor of CSPG receptor is one or more selective antagonist peptide against one or more CSPG receptor selected from the group consisting of: leukocyte common antigen-related (LAR), protein tyrosine phosphatase-sigma (PTPσ), and protein tyrosine phosphatase-delta (PTPδ).

In one embodiment, the one or more inhibitor of one or more CSPG receptor comprises at least three inhibitors of at least three CSPG receptors.

In one embodiment, the at least three inhibitors of at least three CSPG receptors comprise at least three selective antagonist peptides against leukocyte common antigen-related (LAR), protein tyrosine phosphatase-sigma (PTPσ), protein tyrosine phosphatase-delta (PTPδ), or any combination thereof.

In one embodiment, the at least three selective antagonist peptides are expressed as a single peptide separated by self-cleaving 2A peptides.

In one embodiment, one or more of the at least three selective antagonist peptide is conjugated to a sequence that promotes traversal of the peptide across the plasma membrane of a transduced cell into the extracellular space. In one embodiment, the sequence comprises a transactivator of transcription (TAT) sequence (SEQ ID NO: 4).

In one aspect, the present invention relates, in part, to a method of administering an engineered AAV vector to a subject in need thereof comprising: a) obtaining the engineered AAV vector of the present invention; and b) contacting the engineered AAV vector with a cell or tissue of the subject in need thereof.

In another aspect, the present invention relates to a method of promoting regeneration of damaged or degenerated neural tissue in a subject in need thereof comprising: administering to the subject a composition comprising the engineered AAV vector of the present invention. In one embodiment, the subject is a subject having damaged or degenerated neural tissue.

In another aspect, the present invention relates, in part, to a method of treating or preventing one or more disease or disorder associated with damaged or degenerated neural tissue in a subject in need thereof comprising: administering to the subject a therapeutic composition comprising the engineered AAV vector of the present invention. In one embodiment, the disease or disorder associated with damaged or degenerated neural tissue comprises one or more selected from the group consisting of: spinal cord injury, traumatic brain injury, optic neuropathy, stroke, multiple sclerosis, cerebral palsy, acute leukoencephalitis, leukodystrophy, central pontine myelinolysis, Alzheimer's disease, Amyotrophic lateral sclerosis, and Parkinson's disease.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a schematic of the viral capsid of HEBC-AAV9. The drawing shows the mutations and an insertion of peptides into AAV9 capsid protein.

FIG. 2, comprising FIGS. 2A and 2B, depicts exemplary results demonstrating that HEBC-AAV9-Syn vector dramatically enhances transduction efficacy of CNS neurons in adult mice 2 weeks after intravenous injection. FIG. 1A depicts parasagittal brain sections doubly stained for NeuN (neuron marker) and GFP from adult mice treated with 3 versions of AAV9 vectors HEBC-Syn-AAV9-GFP dramatically enhances neuronal transduction efficacy compared with mAAV9-Syn-GFP and AAV-PHP.eB-Syn-GFP (CTX: cortex, HP: hippocampus, TH: thalamus, CB: cerebella, BS: brainstem. Scale: 1 mm). FIG. 1B depicts a graph which indicates the rate of quantified GFP+ transduced cells colocalized with NeuN+ cells in the gray matter of different CNS regions (Student t-test. ** p<0.01).

FIG. 3 depicts exemplary results demonstrating that HEBC-AAV9-GFAP dramatically enhances transduction efficacy of astrocytes in adult mice 3 weeks after intravenous injection. Parasagittal brain sections were stained for GFP in mice treated with AAV-PHP.eB-GFAP-GFP and HEBC-AAV9-GFAP-GFP. The latter transduced most GFAP+ cells in gray matter of different brain regions, in contrast to transducing a portion of astrocytes only in some regions by the former (Scale: 1 mm).

### DETAILED DESCRIPTION

The present invention generally relates to noninvasive and highly effective gene delivery compositions and methods to target specific neural cells in the CNS. The present invention also generally relates compositions and methods for specific and efficient delivery of therapeutic molecules to damaged or degenerated neural tissue to stimulate regeneration.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “antagonist” as used herein in reference to cell receptors refers to a molecule that blocks or dampens a biological response by binding to and blocking a receptor, rather than activating it like an agonist. An antagonist “against” a particular receptor means an antagonist that specifically binds to and blocks said receptor.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

The terms “patient,” “subject,” “individual,” and the like are used interchangeably herein, and refer to any animal or cell whether in vitro or in vivo, amenable to the methods described herein. In one embodiment, the subjects include vertebrates and invertebrates. Invertebrates include, but are not limited to, Drosophila melanogaster and Caenorhabditis elegans. Vertebrates include, but are not limited to, primates, rodents, domestic animals or game animals. Primates include, but are not limited to, chimpanzees, cynomologous monkeys, spider monkeys, and macaques (e.g., Rhesus). Rodents include, but are not limited to, mice, rats, woodchucks, ferrets, rabbits and hamsters. Domestic and game animals include, but are not limited to, cows, horses, pigs, deer, bison, buffalo, feline species (e.g., domestic cat), canine species (e.g., dog, fox, wolf), avian species (e.g., chicken, emu, ostrich), and fish (e.g., zebrafish, trout, catfish and salmon). In some embodiments, the subject is a mammal, e.g., a primate, e.g., a human. In certain non-limiting embodiments, the patient, subject or individual is a human.

The term “engineered AAV vector” means a vector derived from an adeno-associated virus serotype, including without limitation, AAV1, AAV2, AAV3, AAV4, AAV5, AAV6, AAV7, AAV8, and AAV9, and comprising one or more insertion, deletion, or substitution in one or more structural protein.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “inducible” promoter is a nucleotide sequence which, when operably linked with a polynucleotide which encodes or specifies a gene product, causes the gene product to be produced substantially only when an inducer which corresponds to the promoter is present.

A “constitutive” promoter is a nucleotide sequence which, when operably linked with a polynucleotide which encodes or specifies a gene product, causes the gene product to be produced in a cell under most or all physiological conditions of the cell.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

As used herein, “operably linked” sequences include both expression control sequences that are contiguous with a sequence encoding a molecule of interest and expression control sequences that act in trans or at a distance to control the sequence encoding a molecule of interest.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein the term “wild type” is a term of the art understood by skilled persons and means the typical form of an organism, strain, gene or characteristic as it occurs in nature as distinguished from mutant or variant forms.

### Description

The present invention is based, in part, upon the generation of a novel engineered AAV vector with an enhanced ability to traverse the blood-brain barrier (BBB). In some embodiments, the novel engineered AAV vector comprises one or more amino acid insertion or substitution in the viral capsid. In some embodiments, the novel engineered AAV vector comprises a neural cell-specific promoter in the viral genome. In some embodiments, the viral genome encodes one or more inhibitor of neural anti-regenerative pathways. In some embodiments, the novel engineered AAV vector is used to promote regeneration of damaged or degenerated neural tissue. In some embodiments, the novel engineered AAV vector is used to treat or prevent one or more disease or disorder associated with damaged or degenerated neural tissue. In some embodiments, the present invention comprises methods of generating and administering the novel engineered AAV vector of the present invention.

### Engineered AAV Vectors

In one embodiment, the present invention relates to a composition comprising an engineered variant of an adeno-associated virus (AAV) vector (i.e. an engineered AAV vector). AAV vectors have become powerful gene delivery tools for the treatment of various disorders. AAV vectors possess a number of features that render them ideally suited for delivery and expression of DNA encoding therapeutic molecules, including a lack of pathogenicity, minimal immunogenicity, and the ability to transduce postmitotic cells in a stable and efficient manner. Expression of a DNA sequence encoding a therapeutic molecule contained within an AAV vector can be specifically targeted to one or more types of cells by choosing the appropriate combination of AAV serotype, promoter, delivery method.

In some embodiments, the engineered AAV vector can be of a serotype consisting of, but not limited to, serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), and serotype 9 (AAV9). The AAV serotypes demonstrate unique tissue tropisms, as well as other characteristics including differential transduction efficiencies, blood-clearance properties and the ability to traverse the blood-brain barrier. These unique properties appear to be largely dictated by their viral capsid structure.

In one embodiment, the engineered AAV vector comprises an engineered AAV9 vector. In some embodiments, the engineered AAV9 vector comprises one or more amino acid substitution or insertion in the natural (i.e. wild-type) AAV9 viral capsid. In one embodiment, the natural AAV9 viral capsid comprises an amino acid sequence of SEQ ID NO: 1. In one embodiment, the engineered AAV9 vector viral capsid comprises one or more amino acid substitution. In one embodiment, the amino acid substitution comprises a tyrosine-to-phenylalanine substitution at amino acid residue 446. In one embodiment, the amino acid substitution comprises a tyrosine-to-phenylalanine substitution at amino acid residue 731. In one embodiment, the amino acid substitution comprises an alanine-to-aspartic acid substitution at amino acid residue 587. In one embodiment, the amino acid substitution comprises a glutamine-to-glycine substitution at amino acid residue 588. In one embodiment, the viral capsid comprises an insertion of one or more amino acids. In one embodiment, the insertion is at least one, at least two, at least three, at least four, at least five, at least six or at least seven amino acids in length. In one embodiment, the insertion occurs after amino acid residue 588 of the viral capsid. In one embodiment, the insertion comprises seven amino acids. In one embodiment, the seven amino acids comprise threonine, leucine, alanine, valine, proline, phenylalanine and lysine residues. In one embodiment, the seven amino acids comprise the sequence of TLAVPFK (SEQ ID NO: 2).

In one embodiment, the engineered AAV9 vector viral capsid comprises one or more selected from the group consisting of: a tyrosine-to-phenylalanine substitution at amino acid residue 446, tyrosine-to-phenylalanine substitution at amino acid residue 731, an alanine-to-aspartic acid substitution at amino acid residue 587, a glutamine-to-glycine substitution at amino acid residue 588 and a TLAVPFK insertion after amino acid residue 588. In one embodiment, the viral capsid comprises a tyrosine-to-phenylalanine substitution at amino acid residue 446, tyrosine-to-phenylalanine substitution at amino acid residue 731, an alanine-to-aspartic acid substitution at amino acid residue 587, a glutamine-to-glycine substitution at amino acid residue 588 and a TLAVPFK insertion after amino acid residue 588. In one embodiment, the viral capsid comprises an amino acid sequence of SEQ ID NO: 3.

In one embodiment, the engineered AAV vector of the present invention comprises a modified viral genome. AAV vectors can have one or more of the AAV wild-type genes deleted in whole or part (e.g., the rep and/or cap genes), but retain functional flanking inverted terminal repeats (ITRs) that fold into characteristic T-shaped hairpins, which are the only sequences required in cis for the replication and packaging of the AAV genome. In some embodiments, the deleted wild-type genes can be replaced by a sequence that enables expression of one or more therapeutic molecule. In one embodiment, said sequence comprises conventional control elements which are operably linked to a sequence encoding a therapeutic molecule in a manner which permits its transcription, translation and/or expression in a cell transfected with the plasmid vector or infected with the virus produced by the invention.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Promoters can also be used to partially or completely limit expression of a therapeutic molecule of interest to a particular cell-type, and thus reduce or prevent off-target effects of expression in undesired cell-types. In one embodiment of the present invention, the modified viral genome comprises a neural cell-specific promoter. Neural cell-specific promoters include, but are not limited to, synapsin I (Syn) specific for neurons, glial fibrillary acidic protein (GFAP) specific for astrocytes, myelin basic protein (MBP) specific for oligodendrocytes, and CD68 specific for microglia. In one embodiment, the neural cell-specific promoter limits expression to one or more cell selected from the group consisting of, but not limited to, neurons, astrocytes, oligodendrocytes, and microglia. In one embodiment, the neural cell-specific promoter is one or more selected from the group consisting of: Syn, GFAP, MBP, and CD68.

Enhancer sequences found on a vector also regulate expression of the sequence of interest contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In some embodiments of the present invention, the modified viral genome comprises a nucleic acid sequence encoding one or more inhibitor of one or more neural anti-regenerative pathway targets. In some embodiments of the present invention, the modified viral genome comprises a nucleic acid sequence encoding two or more inhibitors of one or more neural anti-regenerative pathway targets. In some embodiments of the present invention, the modified viral genome comprises a nucleic acid sequence encoding three or more inhibitors of one or more neural anti-regenerative pathway targets.

In one embodiment, the neural anti-regenerative pathway target is let-7 miRNA. Let-7 miRNA targets multiple genes that are critical for controlling axon regeneration in CNS. In one embodiment, the inhibitor is a direct let-7 inhibitor. In one embodiment, the direct let-7 inhibitor is an inhibitory RNA. In one embodiment, the inhibitor RNA is a tough decoy (TuD) RNA (anti-let-7 TuD). In one embodiment, the anti-let-7 TuD targets and inhibits all let-7 isoforms. In one embodiment, the anti-let-7 TuD comprises a nucleic acid sequence of AACUAUACAACCAUCUUACUACCUCA (SEQ ID NO: 5) In one embodiment, the inhibitor is a suppressor of let-7 expression. In one embodiment, the suppressor is a protein. In one embodiment, the protein is lin28. In one embodiment, lin28 comprises an amino acid sequence of SEQ ID NO: 6. In one embodiment, the protein is lin41. In one embodiment, lin41 comprises an amino acid sequence of SEQ ID NO: 7.

In one embodiment, the modified viral genome comprises a nucleic acid sequence encoding one or more selected from the group consisting of: anti-let-7 TuD, lin28 and lin41. In one embodiment, the modified viral genome comprises a nucleic acid sequence encoding anti-let-7 TuD, lin28 and lin41. In one embodiment, the nucleic acid sequences encoding the let-7 miRNA inhibitors are separated by nucleic acid sequences encoding 2A peptides. In one embodiment, the 2A peptide comprises an amino acid sequence of GGSGATNFSLLKQAGDVEENPGP (SEQ ID NO: 8). Using 2A peptides allows allow stoichiometric production of multiple peptides by a single vector through a self-cleavage event between the residues of NPG and P in the 2A peptide.

In one embodiment, the neural anti-regenerative pathway target of said inhibitor is one or more receptor of chondroitin sulfate proteoglycans (CSPGs). After CNS injury, reactive glia dramatically upregulate CSPG inhibitors and form a chemical and physical barrier that potently restricts axon elongation. The major receptors for axon growth inhibitors generated by glial scar are leukocyte common antigen-related (LAR), protein tyrosine phosphatase-sigma (PTPσ), and protein tyrosine phosphatase-delta (PTPδ). In one embodiment, said inhibitor is a receptor antagonist. In one embodiment, the receptors targeted by said antagonists are one or more selected from the group consisting of, but not limited to, LAR, PTPσ, and PTPδ. In one embodiment, the antagonist is a selective peptide. In one embodiment, the selective peptide mirrors the sequence of a functional domain of the respective receptor. In one embodiment, the selective antagonist peptide for LAR comprises an amino acid sequence of SEQ ID NO: 9. In one embodiment, the selective antagonist peptide for PTPδ comprises an amino acid sequence of SEQ ID NO: 10. In one embodiment, the selective antagonist peptide for PTPδ comprises an amino acid sequence of SEQ ID NO: 11. In one embodiment, the selective antagonist peptide is conjugated to a sequence that promotes traversal of the peptide across the plasma membrane of a transduced cell into the extracellular space. In on embodiment, said sequence is a transactivator of transcription (TAT) amino acid sequence. In one embodiment, the TAT amino acid sequence comprises the amino acid sequence of GRKKRRQRRRPQ (SEQ ID NO: 4).

In one embodiment, the modified viral genome comprises a nucleic acid sequence encoding one or more selected from the group consisting of: a selective antagonist peptide for LAR, a selective antagonist peptide for PTPσ, and a selective antagonist peptide for PTPδ. In one embodiment, the modified viral genome comprises a nucleic acid sequence encoding a selective antagonist peptide for LAR, a selective antagonist peptide for PTPσ, and a selective antagonist peptide for PTPδ. In one embodiment, the nucleic acid sequence encoding the selective antagonist peptides are separated by nucleic acid sequences encoding 2A peptides. In one embodiment, the 2A peptide comprises an amino acid sequence of SEQ ID NO: 8.

### Methods of Production

In one embodiment, the present invention comprises methods of generating highly efficient, blood-brain barrier traversing engineered AAV vectors of the present invention. In one embodiment, method comprises: 1) transfecting a suitable host cell with one or more nucleic acid comprising the modified AAV viral genome of the present invention and encoding the engineered AAV vector viral capsid, and 2) isolating the packaged engineered AAV viral vector from the suitable host cell. In some embodiments, the method further comprises transfecting said suitable host cell one or more nucleic acid encoding proteins required for AAV replication. In some embodiments, the method further comprises transfecting said suitable host cell one or more nucleic acid encoding one or more adenovirus helper gene.

In some embodiments, the nucleic acid comprising the modified AAV viral genome, the nucleic acid encoding the engineered AAV vector viral capsid, the one or more nucleic acid encoding proteins required for AAV replication, and the one or more nucleic acid encoding one or more adenovirus helper gene are contained within the same vector. In some embodiments, the nucleic acid comprising the modified AAV viral genome is contained within a first vector, the nucleic acid encoding the engineered AAV vector viral capsid and the one or more nucleic acid encoding proteins required for AAV replication are contained within a second vector, and the one or more nucleic acid encoding one or more adenovirus helper gene are contained within a third vector.

Methods of preparing engineered AAV-based vectors and other AAV vectors as are described herein are known. See, e.g., US Published Patent Application No. 2007/0036760, which is incorporated by reference herein. The sequences of any of the AAV capsids provided herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, N.Y.). Alternatively, peptides can also be synthesized by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These and other suitable production methods are within the knowledge of those of skill in the art and are not a limitation of the present invention.

The components required to be cultured in the host cell to package a modified viral genome in an AAV capsid may be provided to the host cell in trans. Alternatively, any one or more of the required components (e.g., modified viral genome, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the modified viral genome. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The modified AAV viral genome, rep sequences, cap sequences, and helper functions required for producing the engineered AAV vector of the invention may be delivered to the packaging host cell in the form of any genetic element which transfer the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating AAV virions are well known and the selection of a suitable method is not a limitation on the present invention. See, e.g., K. Fisher et al, (1993) J. Virol., 70:520-532 and U.S. Pat. No. 5,478,745.

Unless otherwise specified, the AAV ITRs, and other selected AAV components described herein, may be readily selected from among any AAV. These ITRs or other AAV components may be readily isolated using techniques available to those of skill in the art from an AAV sequence. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank®, PubMed®, or the like.

The modified AAV viral genome can be carried on any suitable vector, e.g., a plasmid, which is delivered to a host cell. The plasmids useful in this invention may be engineered such that they are suitable for replication and, optionally, integration in prokaryotic cells, mammalian cells, or both. These plasmids (or other vectors carrying the 5′ AAV ITR-heterologous molecule-3′ AAV ITR) contain sequences permitting replication of the modified AAV viral genome in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the modified AAV viral genome is transfected into the cell, where it may exist transiently. Alternatively, the modified AAV viral genome (carrying the 5′ AAV ITR-heterologous molecule-3′ ITR) may be stably integrated into the genome of the host cell, either chromosomally or as an episome. In certain embodiments, the modified AAV viral genome may be present in multiple copies, optionally in head-to-head, head-to-tail, or tail-to-tail concatamers. Suitable transfection techniques are known and may readily be utilized to deliver the minigene to the host cell.

### Methods of Administration

In one embodiment, the present invention comprises methods of administering the engineered AAV vectors of the present invention to a subject in need thereof. In some embodiments, the method comprises: 1) obtaining the engineered AAV viral vector of the present invention, and 2) contacting said engineered AAV viral vector with a cell or tissue of the subject in need thereof.

The above-described engineered AAV vectors may be delivered to host cells according to published methods. The engineered AAV vector, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the engineered AAV vector and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The engineered AAV vector is administered in sufficient amounts to transfect the cells and to provide sufficient levels of therapeutic molecule expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the lung, heart, or brain), oral, inhalation, intranasal, intratracheal, intraarterial, intraocular, intravenous, intramuscular, subcutaneous, intradermal, and other parental routes of administration. Routes of administration may be combined, if desired.

### Methods of Treatment

In some embodiments, the present invention comprises methods of promoting regeneration of damaged or degenerated neural tissue in a subject in need thereof. In some embodiments, the method comprises administering to a subject having damaged or degenerated tissue a composition comprising the engineered AAV vector of the present invention.

In some embodiments, the composition is administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, the viral genome (vg) concentration of the composition that is administered is between 1.0×10¹¹ vg per kilogram (kg) and 1.0×10¹⁶ vg/kg. In some cases, the concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, or 2×10¹⁷. In some cases the concentration of the infectious particles 3×10⁷, 3×10⁸, 3×10⁹, 3×10¹⁰, 3×10¹¹, 3×10¹², 3×10¹³, 3×10¹⁴, 3×10¹⁵, 3×10¹⁶, or 3×10¹⁷. In some cases the concentration of the infectious particles 4×10⁷, 4×10⁸, 4×10⁹, 4×10¹⁰, 4×10¹¹, 4×10¹², 4×10¹³, 4×10¹⁴, 4×10¹⁵, 4×10¹⁶, or 4×10¹⁷. In some cases the concentration of the infectious particles 5×10⁷, 5×10⁸, 5×10⁹, 5×10¹⁰, 5×10¹¹, 5×10¹², 5×10¹³, 5×10¹⁴, 5×10¹⁵, 5×10¹⁶, or 5×10¹⁷. In some cases the concentration of the infectious particles 6×10⁷, 6×10⁸, 6×10⁹, 6×10¹⁰, 6×10¹¹, 6×10¹², 6×10¹³, 6×10¹⁴, 6×10¹⁵, 6×10¹⁶, or 6×10¹⁷. In some cases the concentration of the infectious particles 7×10⁷, 7×10⁸, 7×10⁹, 7×10¹⁰, 7×10¹¹, 7×10¹², 7×10¹³, 7×10¹⁴, 7×10¹⁵, 7×10¹⁶, or 7×10¹⁷. In some cases the concentration of the infectious particles 8×10⁷, 8×10⁸, 8×10⁹, 8×10¹⁰, 8×10¹¹, 8×10¹², 8×10¹³, 8×10¹⁴, 8×10¹⁵, 8×10¹⁶, or 8×10¹⁷. In some cases the concentration of the infectious particles 9×10⁷, 9×10⁸, 9×10⁹, 9×10¹⁰, 9×10¹¹, 9×10¹², 9×10¹³, 9×10¹⁴, 9×10¹⁵, 9×10¹⁶, or 9×10¹⁷.

In some embodiments, the administering of step is performed once. Alternatively, the administering of step is repeated at least twice. The administering of step may be performed once daily. In some cases, the administering of step comprises intravenous administration. In some cases, the administering comprises pulmonary administration. In some cases, the administering comprises intranasal administration (such as a spray). In some cases, the administering of step comprises injecting the composition into a target in vivo environment. In some cases, the administering of step does not comprise injecting the composition into the target in vivo environment.

In some embodiments, the subject is a mammal. Non-limiting examples of a mammal include a mouse, rat, guinea pig, rabbit, chimpanzee, or farm animal. In some instances, the mammal is a non-human primate. In some instances, the subject is human. The subject of the present disclosure may not be diagnosed with a disease or condition. Alternatively, the subject may be a patient that is diagnosed with a disease or disorder, or suspected of having the disease or the disorder.

In some embodiments, the present invention comprises methods of treating or preventing one or more disease or disorder associated with damaged or degenerated neural tissue in a subject. In some embodiments, the method comprises administering to a subject having a disease or disorder associated with damaged or degenerated neural tissue a composition comprising the engineered AAV vector of the present invention.

In some embodiments, the disease or disorder comprises a disease or condition of the central nervous system (CNS). Non-limiting examples of diseases or disorders of the CNS include: Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Charcot-Marie-Tooth syndrome, classical rhizomelic chondrodysplasia punctata (RCDP), Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, Deafness, De Morsier's Syndrome, Dejerine-Klumpke Palsy, Dementia, Dementia—Multi—Infarct, Dementia—Semantic, Dementia—Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Duchenne muscular dystrophy, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, glioblastoma, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy—Congenital, Myopathy—Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry—Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, and X-Linked Spinal and Bulbar Muscular Atrophy.

In some embodiments, the disease or disorder comprises one or more selected from the group consisting of, but not limited to: spinal cord injury, traumatic brain injury, optic neuropathy, stroke (including brain ischemia and hemorrhage), multiple sclerosis, white matter diseases (e.g., cerebral palsy, acute leucoencephalitis, leukodystrophies, and central pontine myelinolysis), and other axon damage disorders (e.g., Alzheimer's disease, Amyotrophic lateral sclerosis, and Parkinson's disease).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Design of the Highly Efficient BBB-Crossing AAV9 (HEBC-AAV9) Vectors

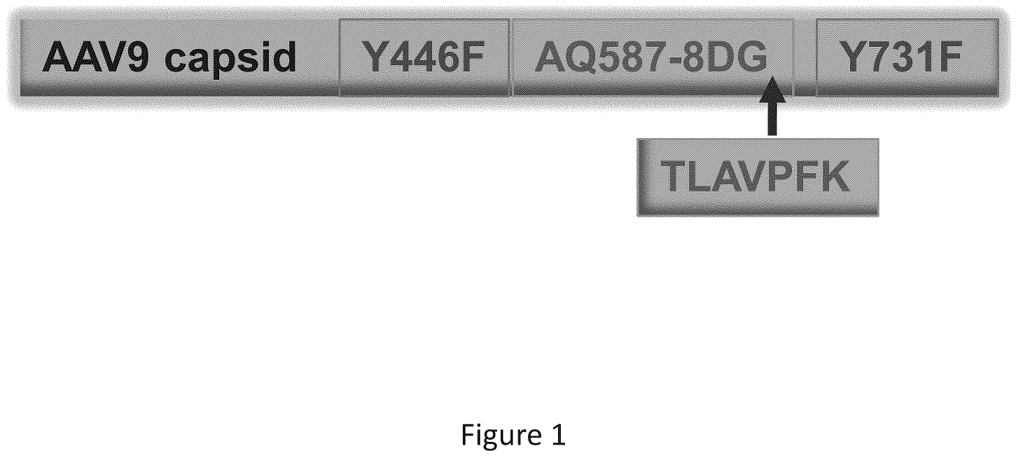

This disclosure claims novel AAV vectors that efficiently transduce targeted neural cells following intravenous (IV) injections. AAV capsid proteins have a highly conserved eight-stranded β-barrel core and expansive loops with connected β-strands and unique surface features. Because most natural AAV serotypes fail to cross BBB in adult mammals, several mutant AAVs have been generated by engineering capsid proteins. However, many show limited efficiency when used at high doses or have species limitations. Thus, highly efficient BBB-crossing AAV9 (HEBC-AAV9) vectors were generated that contain multiple features, aiming to identify the efficiency and potential synergy of combined capsid modifications. The modifications of these new AAV vectors include mutating two surface-exposed Y (tyrosines) to F (phenylalanines) at 446 and 731, mutating AQ (alanine-glutamine) to DG (aspartic acid-glycine) at 587-88 and following insertion of a 7-mer peptide (TLAVPFK) (FIG. 1). HEBC-AAV9 vectors with the cell-specific promoters were also generated for transducing neurons, astrocytes, oligodendrocytes, and microglia specifically in various regions of the nervous system.

### Example 2: Systemic HEBC-AAV9 Vectors Dramatically Show Synergistic Actions on Transducing CNS Cells in Adult Mice

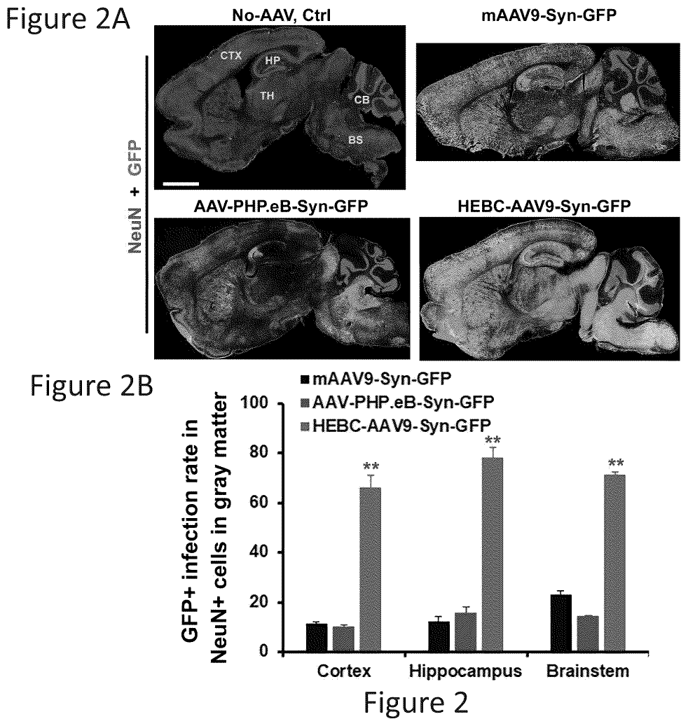

Transduction efficacy of the novel HSBC-AAV9-Syn-GFP was compared with two additional engineered AAV9 vectors (mAAV9-Syn-GFP and AAV-PHP.eB-CMV-GFP) 2 weeks after injecting each of them (50 μl/mouse, 2×10E¹² GC/ml, IV) into 10-week-old C57BL/6 mice, but the same synapsin I (Syn) promoter is used in these vectors. mAAV9-Syn-GFP and AAV-PHP.eB-Syn-GFP transduced a portion of neurons in the brain, but HEBC-AAV9-Syn-GFP transduced the much greater numbers of neurons in all brain regions (by 3-7-fold), including the cortex (especially layer III-V), thalamus, hippocampus, and brainstem (FIG. 2). All GFP signals are colocalized with the neuronal marker NeuN in these mice because the neuronal promoter Syn was used.

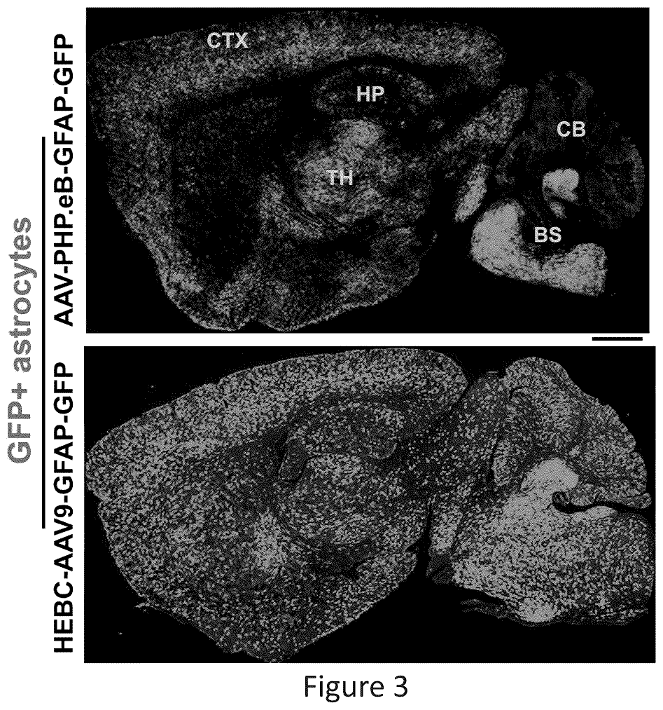

Similarly, HEBC-AAV9-GFAP-GFP vector was evaluated in adult mice (50 μl/mouse, 2×10E¹² GC/ml) 2 weeks after IV injection to transduce astrocytes. Consistently, this vector transduced most GFAP+ astrocytes in the gray matter of various brain regions and spinal cord (79-90%, bottom panel of FIG. 3). HEBC-AAV9-GFAP also transduced both normal and reactive astrocytes around the lesion in the injured spinal cord of mice. In contrast, AAV-PHP.eB-GFAP-GFP only transduced a portion of GFAP+ cells in the gray matter of selected CNS regions (top panel in FIG. 3). For unknown reasons, HEBC-AAV9-GFAP-GFP infected a small portion of GFAP+ astrocytes in white matter whereas AAV-PHP.eB-GFAP transduced almost none. Therefore, systemic HSBC-AAV9 vectors exhibit dramatically increased transduction efficacy for target CNS neural cells in adult mammals.

### Example 3: Sequences

Protein sequence of natural AAV9 capsid
(SEQ ID NO: 1):
MAADGYLPDWLEDNLSEGIREWWALKPGAPQPKANQQHQDNARGL
VLPGYKYLGPGNGLDKGEPVNAADAAALEHDKAYDQQLKAGDNPY
LKYNHADAEFQERLKEDTSFGGNLGRAVFQAKKRLLEPLGLVEEA
AKTAPGKKRPVEQSPQEPDSSAGIGKSGAQPAKKRLNFGQTGDTE
SVPDPQPIGEPPAAPSGVGSLTMASGGGAPVADNNEGADGVGSSS
GNWHCDSQWLGDRVITTSTRTWALPTYNNHLYKQISNSTSGGSSN
DNAYFGYSTPWGYFDFNRFHCHFSPRDWQRLINNNWGFRPKRLNF
KLFNIQVKEVTDNNGVKTIANNLTSTVQVFTDSDYQLPYVLGSAH
EGCLPPFPADVFMIPQYGYLTLNDGSQAVGRSSFYCLEYFPSQML
RTGNNFQFSYEFENVPFHSSYAHSQSLDRLMNPLIDQYLYYLSKT
INGSGQNQQTLKFSVAGPSNMAVQGRNYIPGPSYRQQRVSTTVTQ
NNNSEFAWPGASSWALNGRNSLMNPGPAMASHKEGEDRFFPLSGS
LIFGKQGTGRDNVDADKVMITNEEEIKTTNPVATESYGQVATNHQ
SAQAQAQTGWVQNQGILPGMVWQDRDVYLQGPIWAKIPHTDGNFH
PSPLMGGFGMKHPPPQILIKNTPVPADPPTAFNKDKLNSFITQYS
TGQVSVEIEWELQKENSKRWNPEIQYTSNYYKSNNVEFAVNTEGV
YSEPRPIGTRYLTRNL
AAV9 7-mer insertion peptide sequence
(SEQ ID NO: 2):
TLAVPFK
Protein sequence of HEBC-AAV9 capsid
(SEQ ID NO: 3):
MAADGYLPDWLEDNLSEGIREWWALKPGAPQPKANQQHQDNARGL
VLPGYKYLGPGNGLDKGEPVNAADAAALEHDKAYDQQLKAGDNPY
LKYNHADAEFQERLKEDTSFGGNLGRAVFQAKKRLLEPLGLVEEA
AKTAPGKKRPVEQSPQEPDSSAGIGKSGAQPAKKRLNFGQTGDTE
SVPDPQPIGEPPAAPSGVGSLTMASGGGAPVADNNEGADGVGSSS
GNWHCDSQWLGDRVITTSTRTWALPTYNNHLYKQISNSTSGGSSN
DNAYFGYSTPWGYFDFNRFHCHFSPRDWQRLINNNWGFRPKRLNF
KLFNIQVKEVTDNNGVKTIANNLTSTVQVFTDSDYQLPYVLGSAH
EGCLPPFPADVFMIPQYGYLTLNDGSQAVGRSSFYCLEYFPSQML
RTGNNFQFSYEFENVPFHSSYAHSQSLDRLMNPLIDQYLYFLSKT
INGSGQNQQTLKFSVAGPSNMAVQGRNYIPGPSYRQQRVSTTVTQ
NNNSEFAWPGASSWALNGRNSLMNPGPAMASHKEGEDRFFPLSGS
LIFGKQGTGRDNVDADKVMITNEEEIKTTNPVATESYGQVATNHQ
SDGTLAVPFKAQAQTGWVQNQGILPGMVWQDRDVYLQGPIWAKIP
HTDGNFHPSPLMGGFGMKHPPPQILIKNTPVPADPPTAFNKDKLN
SFITQYSTGQVSVEIEWELQKENSKRWNPEIQYTSNYYKSNNVEF
AVNTEGVYSEPRPIGTRFLTRNL
HIV transactivator of transcription (TAT)
peptide sequence (SEQ ID NO: 4):
GRKKRRQRRRPQ
Anti-let-7 TuD nucleic acid sequence
(SEQ ID NO: 5):
AACUAUACAACCAUCUUACUACCUCA
Lin28 amino acid sequence (SEQ ID NO: 6):
MGSVSNQQFAGGCAKAAEKAPEEAPPDAARAADEPQLLHGAGICK
WFNVRMGFGFLSMTARAGVALDPPVDVFVHQSKLHMEGFRSLKEG
EAVEFTFKKSAKGLESIRVTGPGGVFCIGSERRPKGKNMQKRRSK
GDRCYNCGGLDHHAKECKLPPQPKKCHFCQSINHMVASCPLKAQQ
GPSSQGKPAYFREEEEEIHSPALLPEAQN
Lin41 amino acid sequence (SEQ ID NO: 7):
MASFPETDFQICLLCKEMCGSPAPLSSNSSASSSSSQTSTSSAGG
GGPGAAARRLHVLPCLHAFCRPCLEAHRLPAPGGAGPAEALKLRC
PVCDQKVVLAEAAGMDALPSSAFLLSNLLDAVVATAEEPPPKNGR
AGGGPGGAGGHSNHRHHAHHPAQRAAAPAPQPPPGPAASPGSLLM
RRPHGCSSCDEGNAASSRCLDCQEHLCDNCVRAHQRVRLTKDHYI
ERGPPGPAAASAAQQLGLGPPFAGAPFSILSVFPERLGFCQHHDD
EVLHLYCDTCSVPICRECTLGRHGGHSFAYLQDALQDSRALTIQL
LADAQQGRQALQLSIEQAQTVAEQVEMKAKVVQSEVKAVTARHKK
ALEDRECELLWKVEKIRQVKAKSLFLQVEKLRQSLSKLESTISAV
QQVLEEGRALDILLARDRMLAQVQELKTIRGLLQPQEDDRIMFTP
PDQALYLALKSIGFVSSGAFAPLTKATGDGIKRALQGKVASFTVM
GYDHDGEPRHSGGDLMSVVVLGPDGNLFGAEVSDQQNGTYIVSYR
PQLEGEHLVSVTLYNQHIENSPFKVVVKSGRSYVGIGLPGLSFGS
EGDGEGKLCRPWGVSVDKEGFIIVADRSNNRIQVFKPCGSFHHKF
GTLGSRPGQFDRPAGVACDASRRIIVADKDNHRIQIFTFEGQFLL
KFGEKGTKNGQFNYPWDVAVNSEGKILVSDTRNHRIQLFGPDGVF
LNKYGFEGSLWKHFDSPRGVAFNNEGHLVVTDENNHRLLVIHPDC
QSARFLGSEGSGNGQFLRPQGVAVDQEGRIIVADSRNHRVQMFEA
NGSFLCKFGAQGSGFGQMDRPSGIAVTPDGLIVVVDFGNNRILIF
2A peptide amino acid sequence (SEQ ID NO: 8)
GGSGATNFSLLKQAGDVEENPGP
LAR antagonist peptide amino acid sequence
(SEQ ID NO: 9):
KPRITWMKKGKKVSSQRGGSGETTISGLTPETTYSITVAAYTTKG
DGARSKPKVVTTTGGSGDLADNIERLKANDGLKFSQEYESI
PTPσ antagonist peptide amino acid sequence
(SEQ ID NO: 10):
KPRVTWNKKGKKVNSQRGGSGEMVITNLQPETAYSITVAAYTMKG
DGARSKPKVVVTKGGSGDMAEHMERLKANDSLKLSQEYESI
PTPδ antagonist peptide amino acid sequence
(SEQ ID NO: 11):
RPKIVWNKKGKKVSNQRGGSGDMIISGLQPETSYSLTVTAYTTKG
DGARSKPKLVSTTGGSGELADHIERLKANDNLKFSQEYESI

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An engineered adeno-associated viral (AAV) vector comprising:
a) an engineered AAV viral capsid, wherein the engineered AAV viral capsid comprises at least one selected from the group consisting of: a phenylalanine at amino acid residue 446 of wild-type AAV9 capsid protein VP1, a phenylalanine at amino acid residue 731 of wild-type AAV9 capsid protein VP1, an aspartic acid at amino acid residue 587 of wild-type AAV9 capsid protein VP1, a glycine at amino acid residue 588 of wild-type AAV9 capsid protein VP1, and a TLAVPFK (SEQ ID NO: 2) insertion after amino acid residue 588 of wild-type AAV9 capsid protein VP1; and
b) a modified viral genome comprising a nucleic acid sequence encoding one or more therapeutic molecule.

2. The engineered AAV vector of claim 1, where the wild-type AAV9 capsid protein VP1 comprises an amino acid sequence of SEQ ID NO: 1.

3. The engineered AAV vector of claim 1, wherein the engineered AAV viral capsid comprises an amino acid sequence of SEQ ID NO: 3.

4. The engineered AAV vector of claim 1, wherein the modified viral genome further comprises a neural cell-specific promoter.

5. The engineered AAV vector of claim 5, wherein the neural cell-specific promoter limits expression to one or more type of cell selected from the group consisting of: neurons, astrocytes, oligodendrocytes, and microglia.

6. The engineered AAV vector of claim 6, wherein the neural cell-specific promoter is one or more selected from the group consisting of: synapsin I (Syn), glial fibrillary acidic protein (GFAP), myelin basic protein (MBP), and CD68.

7. The engineered AAV vector of claim 1, wherein the one or more therapeutic molecule comprises one or more inhibitor of one or more neural anti-regenerative pathway targets.

8. The engineered AAV vector of claim 7, wherein the one or more neural anti-regenerative pathway targets is one or more selected from the group consisting of: let-7 miRNA, and one or more receptor of chondroitin sulfate proteoglycans (CSPGs).

9. The engineered AAV vector of claim 8, wherein the one or more inhibitor of let-7 miRNA is one or more selected from the group consisting of: anti-let-7 tough decoy (TuD) RNA (anti-let-7 TuD), lin28, and lin41.

10. The engineered AAV vector of claim 8, wherein the one or more inhibitor of one or more receptor of CSPG receptor is one or more selective antagonist peptide against one or more CSPG receptor selected from the group consisting of: leukocyte common antigen-related (LAR), protein tyrosine phosphatase-sigma (PTPσ), and protein tyrosine phosphatase-delta (PTPδ).

11. The engineered AAV vector of claim 8, wherein the one or more inhibitor of one or more CSPG receptor comprises at least three inhibitors of at least three CSPG receptors.

12. The engineered AAV vector of claim 11, wherein the at least three inhibitors of at least three CSPG receptors comprise at least three selective antagonist peptides against leukocyte common antigen-related (LAR), protein tyrosine phosphatase-sigma (PTPσ), protein tyrosine phosphatase-delta (PTPδ), or any combination thereof.

13. The engineered AAV vector of claim 12, further wherein the at least three selective antagonist peptides are expressed as a single peptide separated by self-cleaving 2A peptides.

14. The engineered AAV vector of claim 13, further wherein one or more selective antagonist peptide of the at least three selective antagonist peptides is conjugated to a sequence that promotes traversal of the peptide across the plasma membrane of a transduced cell into the extracellular space.

15. The engineered AAV vector of claim 14, wherein the sequence comprises a transactivator of transcription (TAT) sequence (SEQ ID NO: 4).

16. A method of administering an engineered AAV vector to a subject in need thereof, the method comprising:
a) obtaining the engineered AAV vector of claim 1; and
b) contacting the engineered AAV vector with a cell or tissue of the subject in need thereof.

17. A method of promoting regeneration of damaged or degenerated neural tissue in a subject in need thereof, the method comprising:
administering to the subject a composition comprising the engineered AAV vector of claim 1.

18. The method of claim 17, wherein the subject is a subject having damaged or degenerated neural tissue.

19. A method of treating or preventing one or more disease or disorder associated with damaged or degenerated neural tissue in a subject in need thereof comprising:
administering to said subject a therapeutic composition comprising the engineered AAV vector claim 1.

20. The method of claim 19, wherein the disease or disorder associated with damaged or degenerated neural tissue is one or more selected from the group consisting of: spinal cord injury, traumatic brain injury, optic neuropathy, stroke, multiple sclerosis, cerebral palsy, acute leukoencephalitis, leukodystrophy, central pontine myelinolysis, Alzheimer's disease, Amyotrophic lateral sclerosis, and Parkinson's disease.


SEQUENCE LISTING (11 sequences)

SEQ ID NO: 1 - protein, 736 aa, Artificial Sequence
  Protein sequence of natural AAV9 capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 7 aa, Artificial Sequence
  AAV9 7-mer insertion peptide sequence
TLAVPFK

SEQ ID NO: 3 - protein, 743 aa, Artificial Sequence
  Protein sequence of HEBC-AAV9 capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYFLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRFLTRNL

SEQ ID NO: 4 - protein, 12 aa, Artificial Sequence
  HIV transactivator of transcription (TAT) peptide sequence
GRKKRRQRRRPQ

SEQ ID NO: 5 - DNA, 26 bp, Artificial Sequence
  Anti-let-7 TuD nucleic acid sequence
aacuauacaaccaucuuacuaccuca

SEQ ID NO: 6 - protein, 209 aa, Artificial Sequence
  Lin28 amino acid sequence
MGSVSNQQFAGGCAKAAEKAPEEAPPDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKNMQKRRSKGDRCYNCGGLDHHAKECKLPPQPKKCHFCQSINHMVASCPLKAQQGPSSQGKPAYFREEEEEIHSPALLPEAQN

SEQ ID NO: 7 - protein, 855 aa, Artificial Sequence
  Lin41 amino acid sequence
MASFPETDFQICLLCKEMCGSPAPLSSNSSASSSSSQTSTSSAGGGGPGAAARRLHVLPCLHAFCRPCLEAHRLPAPGGAGPAEALKLRCPVCDQKVVLAEAAGMDALPSSAFLLSNLLDAVVATAEEPPPKNGRAGGGPGGAGGHSNHRHHAHHPAQRAAAPAPQPPPGPAASPGSLLMRRPHGCSSCDEGNAASSRCLDCQEHLCDNCVRAHQRVRLTKDHYIERGPPGPAAASAAQQLGLGPPFAGAPFSILSVFPERLGFCQHHDDEVLHLYCDTCSVPICRECTLGRHGGHSFAYLQDALQDSRALTIQLLADAQQGRQALQLSIEQAQTVAEQVEMKAKVVQSEVKAVTARHKKALEDRECELLWKVEKIRQVKAKSLFLQVEKLRQSLSKLESTISAVQQVLEEGRALDILLARDRMLAQVQELKTIRGLLQPQEDDRIMFTPPDQALYLALKSIGFVSSGAFAPLTKATGDGIKRALQGKVASFTVMGYDHDGEPRHSGGDLMSVVVLGPDGNLFGAEVSDQQNGTYIVSYRPQLEGEHLVSVTLYNQHIENSPFKVVVKSGRSYVGIGLPGLSFGSEGDGEGKLCRPWGVSVDKEGFIIVADRSNNRIQVFKPCGSFHHKFGTLGSRPGQFDRPAGVACDASRRIIVADKDNHRIQIFTFEGQFLLKFGEKGTKNGQFNYPWDVAVNSEGKILVSDTRNHRIQLFGPDGVFLNKYGFEGSLWKHFDSPRGVAFNNEGHLVVTDFNNHRLLVIHPDCQSARFLGSEGSGNGQFLRPQGVAVDQEGRIIVADSRNHRVQMFEANGSFLCKFGAQGSGFGQMDRPSGIAVTPDGLIVVVDFGNNRILIF

SEQ ID NO: 8 - protein, 23 aa, Artificial Sequence
  2A peptide amino acid sequence
GGSGATNFSLLKQAGDVEENPGP

SEQ ID NO: 9 - protein, 86 aa, Artificial Sequence
  LAR antagonist peptide amino acid sequence
KPRITWMKKGKKVSSQRGGSGETTISGLTPETTYSITVAAYTTKGDGARSKPKVVTTTGGSGDLADNIERLKANDGLKFSQEYESI

SEQ ID NO: 10 - protein, 86 aa, Artificial Sequence
  PTP-sigma antagonist peptide amino acid sequence
KPRVTWNKKGKKVNSQRGGSGEMVITNLQPETAYSITVAAYTMKGDGARSKPKVVVTKGGSGDMAEHMERLKANDSLKLSQEYESI

SEQ ID NO: 11 - protein, 86 aa, Artificial Sequence
  PTP-delta antagonist peptide amino acid sequence
RPKIVWNKKGKKVSNQRGGSGDMIISGLQPETSYSLTVTAYTTKGDGARSKPKLVSTTGGSGELADHIERLKANDNLKFSQEYESI